(19) United States
(12) Patent Application Publication
(10) Pub. No.: US 2025/0289702 A1
(43) Pub. Date: Sep. 18, 2025

(54) INDIVIDUALIZED DISPENSER

(71) Applicants: Avi IFRAH, Ofakim (IL); Tal SHITRIT, Tel Aviv (IL); Yair YONAS, Tel Aviv (IL)

(72) Inventors: Avi IFRAH, Ofakim (IL); Tal SHITRIT, Tel Aviv (IL); Yair YONAS, Tel Aviv (IL)

(73) Assignee: VH20 LTD, Tel Aviv (IL)

(21) Appl. No.: 18/585,567

(22) Filed: Feb. 23, 2024

(30) Foreign Application Priority Data
May 11, 2022 (WO) PCT/IL2022/050900

(51) Int. Cl. B67D 1/00; B67D 1/08

(52) U.S. Cl. CPC B67D 1/0037; B67D 1/004; B67D 1/0888; B67D 2001/0091

(57) ABSTRACT

A dispenser for dispensing an individualized amount of dietary supplements comprising a plurality of replaceable cartridges, each of which containing a dietary supplement solution and at least two of which containing different dietary supplement solutions; a discharge port of the dispenser; a control system for controllably discharging a predefined dose of a dietary supplement solution, upon demand, from at least one of said cartridges to said discharge port; and means for preventing settling of dietary supplement particles within each of said cartridges.

### FIELD OF THE INVENTION

The present invention relates to the field of dispensers. More particularly, the invention relates to a dispenser for dispensing a liquid having a customized composition.

### BACKGROUND OF THE INVENTION

Water dispensers are known to provide better tasting water than normal tap water by purifying and filtering the available water to remove contaminants, and are also capable of instantly dispensing hot or cold water at a personalized temperature.

Drinking water generally contains many minerals and other inorganic chemicals that provide a significant percentage of the nutrient requirements needed by the human body. For example, drinking water provides up to 20% of the required total daily intake of calcium and magnesium. Food intake for most people supplements the amount of nutrients not provided by drinking water. Some people, however, suffer from a nutrient deficiency if their normal diet does not include certain foods or if their body has trouble absorbing or processing some of these nutrients. A nutrient deficiency may be in terms of a deficiency in one or more vitamins, minerals, fiber, amino acids, herbs, and enzymes.

Consequently, many people take dietary supplements to supplement their nutrient deficiency without the need for a prescription. Since the nutrient deficiency is individualized, many members of a household usually need different dietary supplements, often resulting in a state of disarray due to the number of vitamin types in capsule form used by the household, each of which is held by a relatively large sized vitamin container, and due to limited medicine cabinet space.

It is an object of the present invention to provide a dispenser to dispense an amount of liquid that includes a customized composition of one or more desired dietary supplements.

It is an additional object of the present invention to provide a compact dispenser that is able to produce a customized and repeatable dose of dietary supplements to one or more users upon demand.

Other objects and advantages of the invention will become apparent as the description proceeds.

### SUMMARY OF THE INVENTION

A dispenser for dispensing an individualized amount of dietary supplements comprises a plurality of replaceable cartridges, each of which containing a dietary supplement solution and at least two of which containing different dietary supplement solutions; a discharge port of the dispenser; a control system for controllably discharging a predefined dose of a dietary supplement solution, upon demand, from at least one of said cartridges to said discharge port; and means for preventing settling of dietary supplement particles within each of said cartridges.

The dispenser preferably further comprises a corresponding injector member associated with each of the cartridges for causing discharge of the predefined dose of dietary supplement solution to the discharge port and which is in data communication with the control system. Each of the cartridges may be fitted with a substantially vertically oriented cylindrical barrel prefilled with a specific dietary supplement solution.

In one aspect, each of the cartridges for causing discharge of the predefined dose of dietary supplement solution to the discharge port is driven by a pneumatic means, and wherein the pneumatic means are controlled by an electric faucet.

In one aspect, the corresponding injector member comprises a piston that is sealingly engaged with the barrel, a plunger connected to the piston, and an electromechanical actuator positioned above the barrel to cause plunger displacement.

In one aspect, the electromechanical actuator is a stepper motor in data communication with the control system, and the plunger is a plunger rod downwardly extending from a drive shaft of the stepper motor with which it is connected to the piston, the corresponding injector member further comprising a cylinder surrounding, and in threaded engagement with, the plunger rod to thereby facilitate limited and determinable rotational displacement of the plunger rod and corresponding discharge of the predefined dose of the dietary supplement solution in response to plunger rod rotation.

In one aspect, the means for preventing settling of dietary supplement particles within each of said cartridges comprises stirring means.

In one aspect, the stirring means comprises a rotatable dietary supplement additive unit provided with the plurality of cartridges, such that the centrifugal force acting on all of said cartridges during rotation of said dietary supplement additive unit causes the dietary supplement solution to be sufficiently stirred to prevent settling of the dietary supplement particles.

In one aspect, the dietary supplement additive unit comprises a circular base that is rotatable about a central vertical axis; an electrical motor in driving engagement with said base; the plurality of cartridges that are secured to said base and are circumferentially spaced from each other; and the corresponding injector member associated with each of the cartridges.

In one aspect, a bottom end of the barrel has lower tapered surfaces that taper into a hollow tip from which a conduit extends radially inwardly to the discharge port, such that the predefined dose of the dietary supplement solution is urged to flow through the barrel, conduit and discharge port.

In one aspect, the means for preventing settling of dietary supplement particles within each of said cartridges further comprises a metallic ball that loosely sits on top of the hollow tip and a permanent magnet which is fixedly housed within a casing of the dispenser and adapted to be magnetically coupled with the metallic ball of the cartridge instantaneously rotated to a given region of the casing, causing the metallic ball to become unseated from the hollow barrel tip and also to be rotatably displaced within the barrel by an agitating action.

In one aspect, the dispenser is a water dispenser that further comprises watery delivery apparatus for timely delivering a supply of potable water to the discharge port so that said water supply will be combined with the predefined dose of dietary supplement, and generally temperature control apparatus for regulating the temperature of the supply of water.

In one aspect, the dispenser further comprises means for submitting a request to the control system for a desired dietary supplement transfer operation by which a predefined dose of each of a combination of dietary supplement solutions is discharged to the discharge port.

In one aspect, the means for submitting a request to the control system is a touch screen.

In one aspect, the means for submitting a request to the control system comprises communication apparatus, the communication apparatus configured to receive the request by a signal transmitted from a dedicated application running on a processor of a computerized device.

In one aspect, dietary supplement particles that are prevented from settling within each of the cartridges is powder that is maintained as a dispersed suspension within the dietary supplement solution.

### BRIEF DESCRIPTION OF THE DRAWINGS

In the drawings:

FIG. 1 is a perspective view from the top of an embodiment of an individualized water dispenser;

FIG. 2 is a side view of the water dispenser of FIG. 1;

FIG. 3 is a top view of the water dispenser of FIG. 1;

FIG. 4 is a perspective view from the side of the water dispenser of FIG. 1 when its casing and dietary supplement additive unit shell are made transparent to schematically illustrate the water delivery and temperature control apparatus;

FIG. 5 is a perspective view of a dietary supplement additive unit used in conjunction with the water dispenser of FIG. 1, when its outer shell is removed;

FIG. 6 is a side view of one of the cartridges of the dietary supplement additive unit of FIG. 5, shown when separated from the injector member;

FIG. 7 is a top view of the cartridge of FIG. 6;

FIG. 8 is a cross-sectional view of the dietary supplement additive unit of FIG. 5, cur along plane A-A of FIG. 5;

FIG. 9 is a bottom view of an embodiment of a base used in conjunction with the dietary supplement additive unit of FIG. 5;

FIG. 10 is a perspective view from the top of the base of FIG. 9;

FIG. 11 is a bottom view of another embodiment of a base used in conjunction with the dietary supplement additive unit of FIG. 5, showing additional apparatus housed in the water dispenser casing and without the casing;

FIG. 12 is a top view of the dietary supplement additive unit when each of the cartridges is coupled with the base of FIG. 11;

FIG. 13 is a cross sectional view of the dietary supplement additive unit, cut along plane B-B of FIG. 12;

FIG. 14 is a block diagram of an embodiment of a control system used in conjunction with the water dispenser of FIG. 1 to control and monitor the flow of a dietary supplement;

FIG. 15 is a transparent perspective view of an individualized water dispenser, according to another embodiment of the invention;

FIG. 16A illustrates a perspective view of the dietary supplement additive unit of the water dispenser of FIG. 15, according to an embodiment of the invention;

FIG. 16B illustrates a bottom view of the dietary supplement additive unit of FIG. 16A;

FIG. 16C illustrates a top view of the dietary supplement additive unit of FIG. 16A;

FIG. 16D illustrates a cross-section view of the dietary supplement additive unit of FIG. 16A;

FIG. 16E illustrates another perspective view of the dietary supplement additive unit of FIG. 16, in which one the vitamin capsule is extracted out of the dietary supplement additive unit;

FIG. 17A illustrates a transparent perspective view of the vitamin capsule of FIG. 16E; and

FIG. 17B illustrates a side view of the vitamin capsule of FIG. 17A.

### DETAILED DESCRIPTION OF THE INVENTION

An individualized water dispenser comprises a plurality of replaceable cartridges, each of which containing a different dietary supplement solution. A predefined dose of a dietary supplement, accurately determined by the control system, is discharged from one or more of the cartridges and is able to be mixed with a supply of water, generally mains-supplied water, which is adapted to be temperature regulated and filtered by the water dispenser.

Many dietary supplements are provided in powder form since the amount of a dietary supplement needed for an effective dose often cannot suitably fit in the limited volume of a capsule. To avoid the tendency of dietary supplement powder to settle out of a suspension and then harden, leading to a reduced dose of the dietary supplement being discharged to a drinking vessel, the dietary supplement solution in one embodiment is stirred while located within a relatively small-sized cartridge to maintain a substantially dispersed suspension.

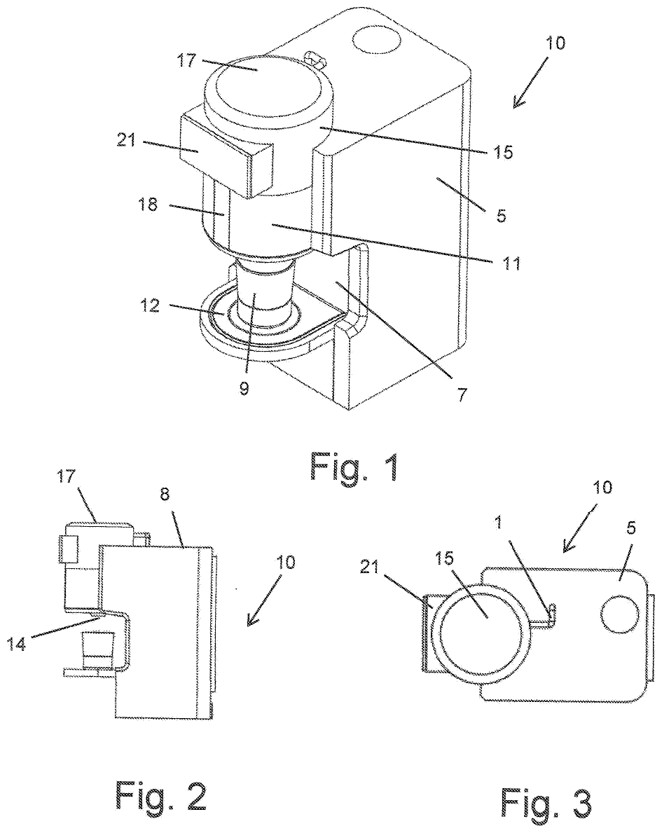

FIGS. 1-3 illustrate an individualized water dispenser 10, according to an embodiment. Water dispenser 10 has a dietary supplement additive unit 15 that can ensure reliable dispensing of a customized composition and dose of a dietary supplement solution that is unique for a specific user. As will be described hereinafter, water dispenser 10 is able to establish a user-specific dietary supplement additive profile in conjunction with a dedicated application to provide the dietary supplement solution.

Water dispenser 10 comprises casing 5, e.g. having a rectilinear configuration, in which are housed water delivery and temperature control apparatus, dietary supplement additive unit 15, and a touch screen 21 by which various settings are able to be entered. The outer shell 11 of dietary supplement additive unit 15 is shown to be cylindrical, and is fixed to the wall of a complementary opening formed in casing 5, such that the upper circular plate 17 of the shell vertically protrudes above the upper surface 8 of casing 5, although other shell configurations are also within the scope of the invention. Touch screen 21, by which all commands for a dietary supplement transfer operation are entered, is fixed to, and radially protrudes from, the periphery of an upper shell portion. Dietary supplement additive unit 15 comprises a plurality of replaceable cartridges, for example, ten cartridges such that each contains one of the following dietary supplements: vitamin A, vitamin B, vitamin C, vitamin D, vitamin E, vitamin K, folic acid, calcium, iron, and zinc. Each of the cartridges is able to be replaced through a door 18 that is pivotally connected to shell 11, and that is, when closed, flush with shell 11.

A drip tray 12 attached to a front vertical wall 7 of casing 5 defines a suitable location of a cup 9 adapted to receive the desired dose of a dietary supplement solution that is gravitationally delivered from discharge port 14 extending downwardly from the bottom of the outer shell. The dispensed dietary supplement solution is generally combined from both the watery delivery apparatus, which discharges a cupful supply of purified and filtered water, and also from the dietary supplement additive unit, which discharges the dose of the customized composition, although the dietary supplement solution may be dispensed without being combined with a supply of water.

Alternatively, the driving vessel adapted to receive the dispensed dietary supplement solution is a bottle. Drip tray 12 may be conveniently folded to accommodate the larger height of a bottle relative to a cup.

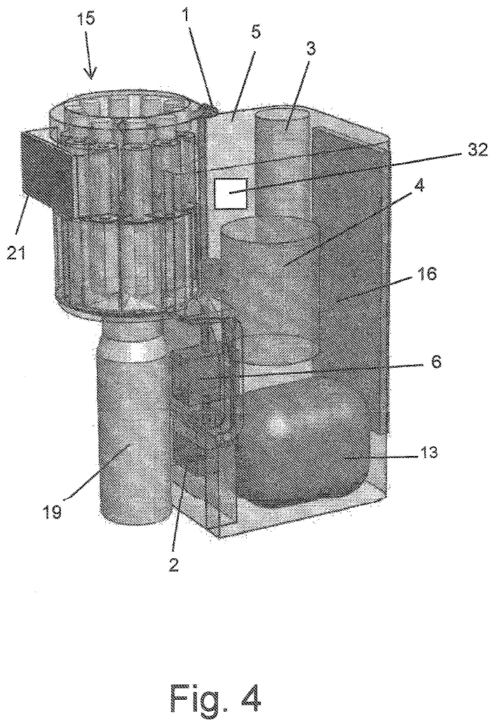

An exemplary configuration of the water delivery and temperature control apparatus housed within casing 5 of water dispenser 10 is shown in FIG. 4.

A supply of water is collected in reservoir 4, after having been introduced to the water dispenser from the water mains and having been filtered and purified by filter 3. Following submission of a request for water, generally in conjunction with a dietary supplement transfer operation, control valve 6 is opened, and a predetermined volume of filtered water flows from reservoir 4 to evaporator 2, in order to be suitably cooled. The chilled water flows upwardly and then laterally and downwardly via angled conduit 1 from control valve 6 to an outlet located at the longitudinal axis of dietary supplement additive unit 15, such that it is able to be mixed with the dietary supplement discharged from one or more of the cartridges.

A low temperature liquid refrigerant flowing in a refrigeration cycle as well known to those skilled in the art absorbs the heat of the water flowing through evaporator 2, and the heated refrigerant vapor exiting evaporator 2 is delivered to compressor 13. The high pressure refrigerant exiting compressor 13 is condensed by condenser 16.

The temperature control apparatus may also comprise a heating element that is operatively connected to another conduit exiting reservoir 4, generally by means of another control valve, and bypassing evaporator 2. The heated water flows to an outlet located at the longitudinal axis of dietary supplement additive unit 15, such that it is able to be mixed with the dietary supplement discharged from one or more of the cartridges and dispensed to bottle 19 when the drip tray is folded.

Control circuitry 32 in data communication with screen 21, generally comprising a processor, and used for effecting desired dietary supplement transfer operations is also housed within casing 5.

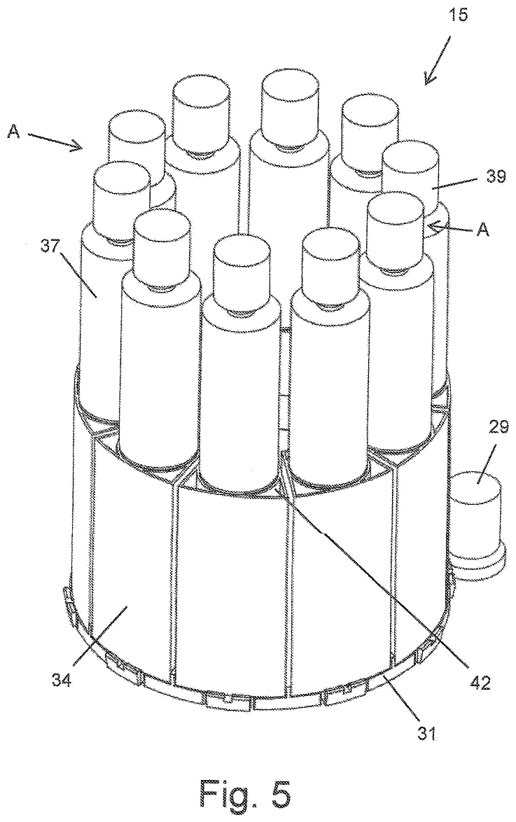

FIG. 5 illustrates the structure of dietary supplement additive unit 15, according to an embodiment, when the outer shell is removed. Dietary supplement additive unit 15 comprises a rotatable carousel to ensure that the dietary supplement provided with each cartridge will be substantially uniformly stirred, as will be explained.

It will be appreciated that a dietary supplement additive unit provided with a plurality of accurately dispensable cartridges may be configured without a rotatable carousel.

Dietary supplement additive unit 15 comprises a circular base 31 that is rotatable about a central vertical axis by means of an electrical motor 29 with which its periphery is in driving engagement through a dedicated aperture formed in the shell of the dietary supplement additive unit and which is located within the casing of the water dispenser. Driving motor 29 may be in constant operation or, alternatively, in intermittent operation, being operated only following a request for a dietary supplement. Positioned on top of base 31 are a plurality of circumferentially spaced cartridges 34, in each of which is fitted a corresponding barrel 42 that is substantially vertically oriented and is prefilled with a specific dietary supplement solution. All barrels 42 of dietary supplement additive unit 15 have a uniform volume, e.g., 30 mL. The radially outer wall of each cartridge 34 is slightly curved, so that the outer wall 46 (FIG. 7) of all the cartridges collectively define a circular shape, similar to that of base 31. Positioned on top of each barrel 42, and in cooperation therewith, is a corresponding injector member 37, each of which comprising a piston that is sealingly engaged with the barrel and an electromechanical actuator 39 for causing plunger displacement.

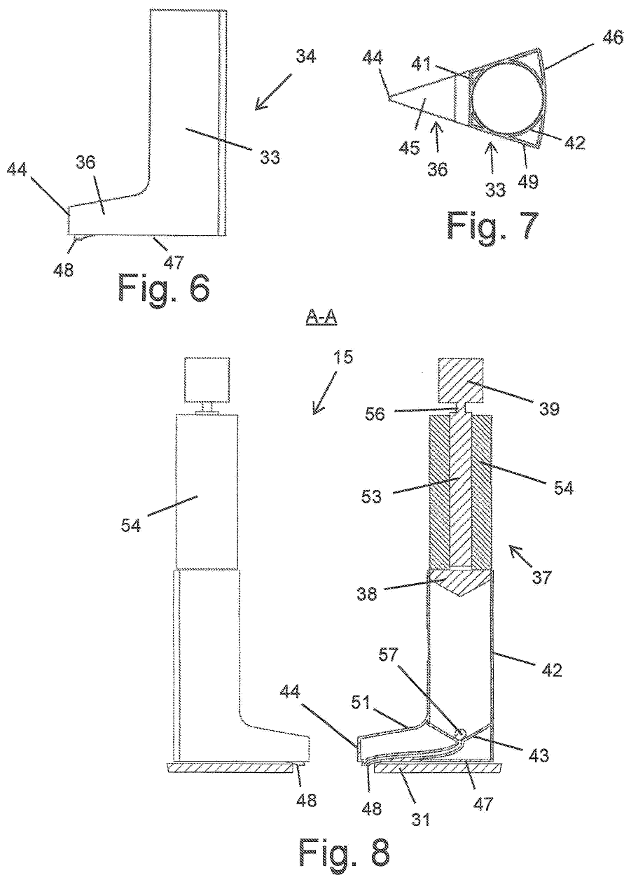

As shown in FIGS. 6 and 7, each cartridge 34 has a radially outward, vertically elongated region 33 with a trapezoidal cross section, in which open-ended cylindrical barrel 42 is fitted. Each side of trapezoidal region 33, including inner wall 41, outer wall 46, and obliquely extending side walls 49, is attached to barrel 42 by a vertical connection. The two side walls 49 continue to extend along a radially inward stabilizing region 36 of the cartridge, which is integrally formed with, and is significantly shorter than, region 33, and terminate at a short common truncated surface 44 that appears as an apex when viewed from above. Region 36 is covered by triangular surface 45. Cartridge 34 also has a bottom surface 47, from which conduit 48 downwardly extends close to truncated surface 44.

FIG. 8 is a cross sectional view of dietary supplement additive unit 15.

Injector member 37 comprises a vertically oriented plunger rod 53 with female threads that is threadedly engageable with the male threads of the surrounding cylinder 54, to ensure precise plunger rod movement and corresponding accurate dispensing of the dietary supplement. In this embodiment, electromechanical actuator 39 is a stepper motor that is capable of rotating plunger rod 53 for only a predetermined fraction of a rotation, or for only a limited number of rotations. The upper end of plunger rod 53 is connected to the drive shaft 56 of stepper motor 39, and its lower end is connected to elastomeric piston 38, which is sealingly and rotatably displaceable within cylindrical barrel 42, so that the predefined dose of the dietary supplement will be accurately dispensed while piston 38 is urged downwardly to displace the dietary supplement solution collected in the barrel in response to plunger rod rotation. Both stepper motor 39 and cylinder 54 are preferably fixedly connected to the shell of the dietary supplement additive unit, such as to the upper plate 17 thereof (FIG. 1).

Barrel 42 has lower tapered surfaces 43 that taper into a hollow tip at the bottom end of the barrel. Attached to one of the tapered surfaces 43 is an integral foot 51 that curves radially inwardly within the stabilizing region of cartridge 34 while being slightly spaced above cartridge bottom surface 47, which is triangular, until protruding radially inwardly from cartridge bottom surface 47 and from base 31 therebelow. Conduit 48 in liquid communication with the hollow barrel tip extends underneath, and is held by, foot 51 until its radial inward end is located proximate to truncated surface 44. A metallic ball 57 loosely sits on top of the hollow tip.

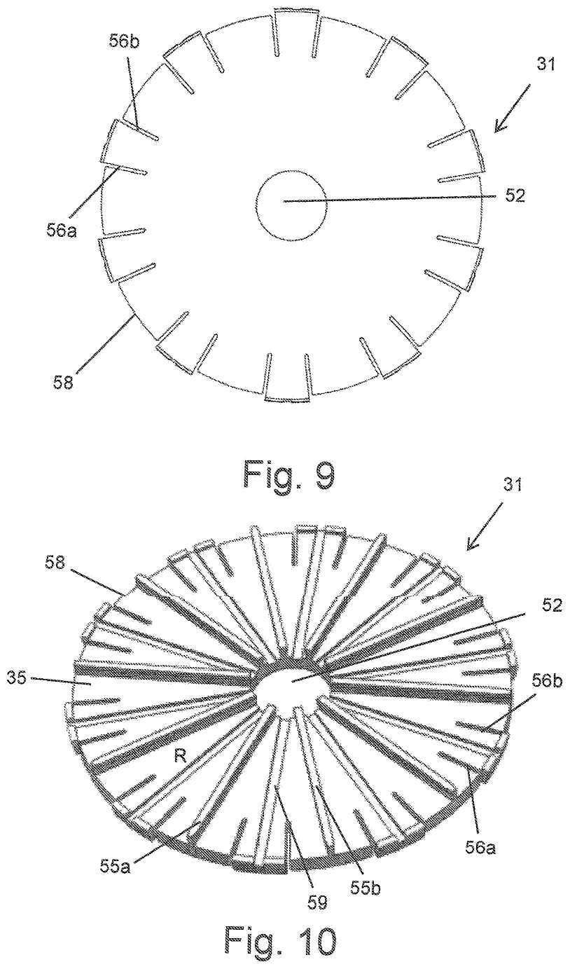

Circular base 31 shown in FIG. 9 has a central cutout 52 within which is positionable a plurality of conduits, one for each cartridge, from which dietary supplement is dischargeable from the corresponding cartridge to discharge port 14 (FIG. 2). Circumferentially spaced pairs of radial slits 56a-b are formed in the periphery 58 of base 31 for a fraction of the radius of base 31, to increase the flexibility of the base when a cartridge is being coupled thereto.

FIG. 10 illustrates a top view of base 31, showing means provided for facilitating the coupling of each cartridge thereto. A pair of radially extending partitions 55a-b that are circumferentially spaced from each other extend from cutout 52 to periphery 58 of base 31, defining the triangular region R in which a corresponding cartridge is positionable and coupleable. For region R, a coupling element 59 is recessed in surface 35 of base 31, and radially extends from cutout 52 to periphery 58 to bisect the angle defined by partitions 58a and 58b and also the angle defined by radial slits 56a and 56b, which are also formed in region R. The triangular cartridge bottom surface is formed with a radially extending protrusion which is adapted to be frictionally received in the corresponding recess 59 when the corresponding cartridge is being coupled to base 31.

Alternatively, the triangular cartridge bottom surface may be formed with a radially extending recess and base surface 35 may be formed with a radially extending protrusion adapted to be received in the recess.

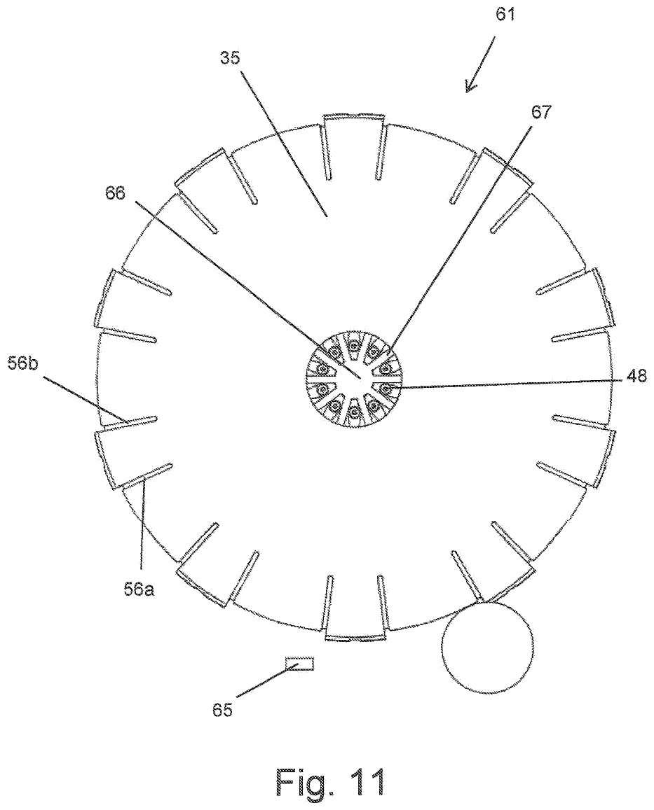

In an embodiment shown in FIGS. 11-13, a base 61 similar to base 31 of FIG. 10 may be additionally configured with a snap 64 that vertically protrudes from the base periphery 58, for each triangular region. A hub 66 with a plurality of circumferentially spaced spokes 67 radially extending from hub 66 is inserted within cutout 52, and is frictionally engaged or integrally formed with, the base surface 35. Each spoke 67 may be aligned with a corresponding partition.

Hub 66 may be configured with an upwardly protruding impediment 69 of a slightly larger diameter and of a slightly larger height than a central vertically protruding hub element 68. With this geometry, a cartridge 34 is forced in one-way engagement with base 61. That is, when truncated surface 44 of cartridge 34 is positioned so as to contact impediment 69 and the two side walls 49 contact partitions 58a-b of a selected region R (FIG. 10), respectively, outer wall 46 of cartridge 34 is forced in engagement with snap 64. Base surface 35 flexes when contacted by cartridge bottom surface 47 through the cooperation of slits 56a-b, and then outer wall 46 is locked in place by snap 64. When all cartridges 34 are coupled to base 61, each corresponding conduit 48 is in abutting relation with a pair of spokes 67 to facilitate discharge of the dietary supplement through the cutout opening 52.

In addition or in replacement, a limit switch or other type of sensor may be used to detect contact between cartridge bottom surface 47 and base 61, indicating that the cartridge has been coupled with the base.

Also shown in FIG. 11 is a permanent magnet 65 that is fixedly housed within the casing of the water dispenser. Permanent magnet 65 is adapted to be magnetically coupled with the metallic ball 57 of the cartridge 34 instantaneously rotated to a given region of the casing, causing the metallic ball to become unseated from the hollow barrel tip and also to be rotatably displaced within the corresponding barrel 42. Consequently, settling of powder within the dietary supplement solution is mitigated, or altogether eliminated, by the centrifugal force acting on all barrels during rotation of dietary supplement additive unit 15 which causes the dietary supplement solution to be gently stirred, and also by the rotatable displacement of metallic ball 57 which causes a more intensive agitating action within a given barrel. Thus hardening of the settled powder which would reduce flow of the dietary supplement through the hollow barrel tip upon operation of the corresponding injector member is also prevented.

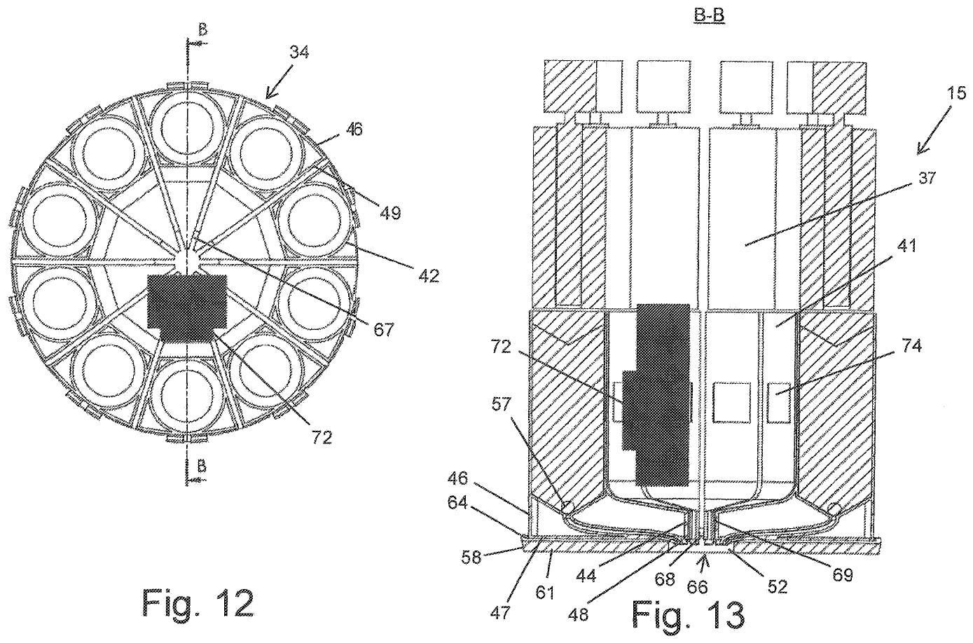

Also shown in FIGS. 12 and 13 is a barcode reader 72, such as a scanner adapted to read a barcode label 74, e.g. a QR code, adhesively attached to, or otherwise affixed to, the cartridge inner wall 41. Barcode reader 72 is positioned within the interior of dietary supplement additive unit 15 and above the cartridge stabilizing regions by means of one or more arms (not shown) that extend at an angle from casing 5 (FIG. 1), so as to be stationary without interfering with any of the injector members 37 when undergoing rotational displacement. Barcode reader 72 is spaced sufficiently close to the barcode label 74 of the cartridge 34 instantaneously rotated to scanning proximity therewith to read the associated data related to barcode label 74. The associated data includes at least a cartridge identifier and the type and composition of the dietary supplement that is included in the cartridge.

Alternatively, each cartridge may be identifiable by means of a corresponding RF tag, such as one that is attached to the inner wall of the cartridge. The RF tag transmits cartridge-specific digital data modulated with a return signal to a reader in response to being interrogated.

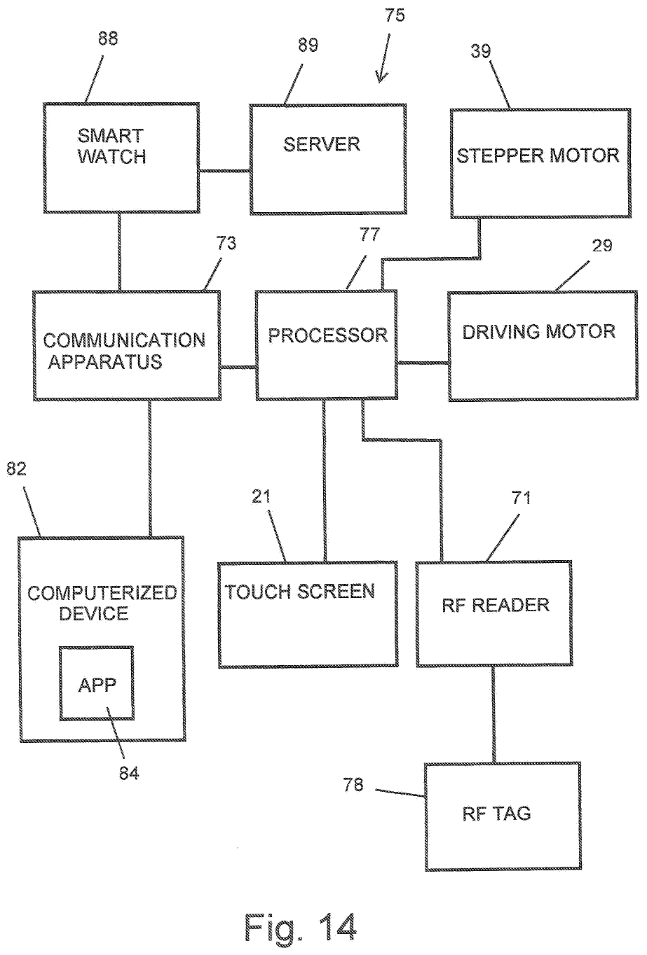

A control system 75 shown in FIG. 14 according to one embodiment is used to control and monitor the flow of a dietary supplement through the dietary supplement additive unit. Processor 77 housed within the water dispenser casing is in data communication with driving motor 29 for rotatably driving the base, each stepper motor 39 of a corresponding injector member, as well as with touch screen 21, such as by Bluetooth or Wi-Fi means. Processor 77 is also in data communication with RF reader 71, housed for example within the water dispenser casing. RF reader 71 in turn is in data communication with each RF tag 78 that is attached to a corresponding cartridge. Communication apparatus 73 connected to processor 77, whether wirelessly or by a wired connection, facilitates data communication with components of the water dispenser as well as with a remote user.

Alternatively, the previously described barcode reader may be in data communication with processor 77 in order to identify each cartridge.

After the user enters a request with touch screen 21 for a dietary supplement transfer operation, such as the dispensing of a single selected dietary supplement of a predetermined dose to be immediately dispensed or to be dispensed at a later predetermined time, or the simultaneous dispensing of a plurality of different dietary supplements, a corresponding signal is transmitted from touch screen 21 to processor 77. In response, processor 77 commands driving motor 29 to be activated so that the base and all of the dietary supplement solutions will be sufficiently stirred. While the base is rotating, processor 77 additionally commands activation of the stepper motor 39 of the injector member adapted to inject the selected dietary supplement through the conduit of the corresponding cartridge in order to be discharged to a drinking vessel. The selected stepper motor is activated for the predetermined time needed to inject the predetermined dose of the selected dietary supplement.

During the course of the dietary supplement transfer operation, processor 77 updates the current device-specific inventory of the dietary supplements. As the maximum volume of dietary supplement that has been prefilled in the barrel of a cartridge is known, and the volume of the dispensed dose of dietary supplement is determined, the inventory is updated with the current remaining volume of the recently dispensed dietary supplement. If a plurality of dietary supplements are dispensed during a transfer operation, the inventory is similarly updated with the current remaining volume of all the recently dispensed dietary supplements.

By updating the current device-specific inventory of dietary supplements, processor 77 is able to determine when the supply of a certain dietary supplement has become depleted, or is about to become depleted, and accordingly notifies the user by a message that is displayed on touch screen 21. After receiving the depletion notification, the user enters via touch screen 21 a request to replace the given cartridge with the depleted dietary supplement. In response, processor 77 commands driving motor 29 to operate for a predetermined duration until the given cartridge with the depleted dietary supplement is positioned proximate to door 18 (FIG. 1). When the given cartridge is positioned proximate to door 18, processor 77 commands the stepper motor 39 associated with the given cartridge to operate in a second rotational direction opposite to the first rotational direction that causes the dietary supplement to be discharged, so that the piston will be upwardly displaced and released from the barrel of the given cartridge. As movement of the given cartridge is no longer restricted by the piston, the given cartridge is able to be removed from the dietary supplement additive unit and subsequently replaced. The upward travel of the plunger rod until the piston is dislodged from the barrel is registered. Consequently, after the user updates processor 77 that a new prefilled cartridge has been coupled to the base, the stepper motor 39 associated with the given cartridge is commanded to operate in the first rotational direction, such that the plunger rod will be downwardly displaced for a predetermined travel that is sufficient to introduce the piston in sealing engagement with the barrel of the replaced cartridge at a beginning discharge position.

Instead of entering a request for a dietary supplement transfer operation or for a cartridge replacement operation with the touch screen, a request may be conveniently entered by means of a dedicated application 82 running on the processor of a computerized device 84, such as a mobile phone, e.g. a smartphone. Various options may be selected through the user interface of the application, such as type and dose of a dietary supplement to be dispensed, and time of day when it is desired that the transfer operation will be performed. Also, it may be requested that the selected dietary supplements be dispensed together with cold water, or alternatively with lukewarm water. For example, a first desired combination of dietary supplements may be dispensed at a first time, and a second desired combination of dietary supplements may be dispensed at a second time during the same 24-hour period. The request may be transmitted remotely from computerized device 84 to the communication apparatus 73 of the given water dispenser, such as via a cellular network. One combination of dietary supplements may be sleep-promoting supplements, and another combination may be an energy booster.

The daily regimen of dietary supplements to be consumed by a user may be recommended by a dietician, and subsequently converted to a profile by built-in instructions provided by application 82. Following interaction of application 82 with the processor 77 of a given water dispenser, whether one regularly accessed by the user or any other water dispenser selected by the user, one or more dietary supplement transfer operations initiated by application derived commands will be performed, whether on a daily basis or through any other regular schedule.

it will be appreciated that one or more dietary supplement transfer operations based on dietician recommendations to be performed on a daily basis or during any other regular schedule may be initiated by touch screen commands, whether on a daily basis or through any other regular schedule.

Alternatively, the daily regimen of dietary supplements to be consumed by a user may be determined by a smartwatch 88 or other suitable device worn by the user. Smartwatch 88, which may be in data communication with the communication apparatus of a selected water dispenser or of a remote server 89, may conduct biomonitoring, such as by detecting various health indicators of the user over a long period of time and transmitting data representative of the health indicators over a signal. Exemplary health indicators include blood pressure, heart rate, and the chemical makeup of sweat droplets to help determine the metabolism and nutrient level of the user's body. A suitable algorithm provided with server 89, for example, is configured to analyze the received health indicator data and to conveniently output a recommended schedule of dietary supplement intake.

According to an embodiment of the invention, remote server 89 receives medical data associated with a user, analyzes the received data, and sends the analysis to the application to provide assessment for one or more dietary supplement transfer operations. For example, remote server 89 may receive data of blood testing results that accordingly may help tailor a dietary supplement transfer operation based on these results. The remote server, application, and the dispenser work together as an “ecosystem” that enables to dispense of a customized composition and dose of a dietary supplement solution that is unique for a specific user (e.g., based on user's actual medical condition, daily activities, or any other available data source).

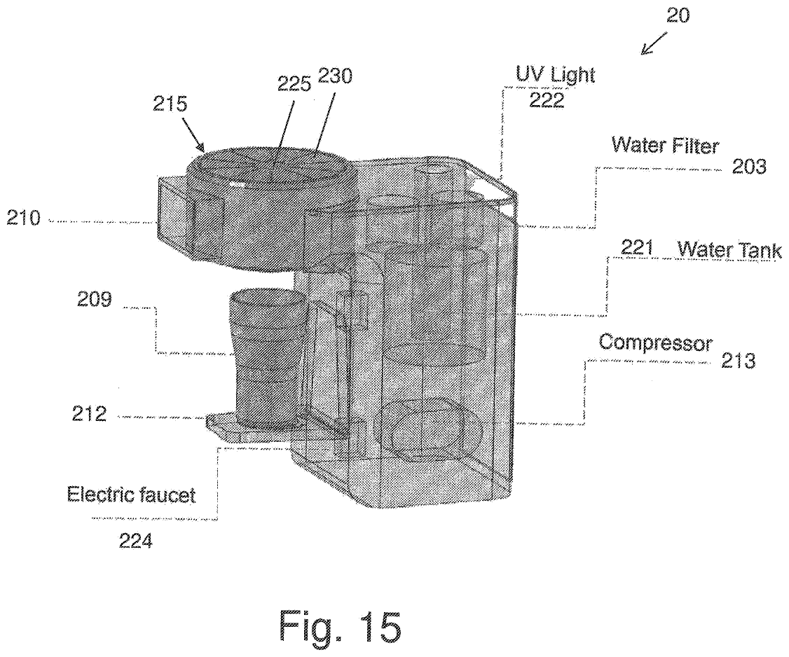

FIG. 15 illustrates an individualized water dispenser 20, according to another embodiment. Water dispenser 20 has a dietary supplement additive unit 215 that can ensure reliable dispensing of a customized composition and dose of a dietary supplement solution that is unique for a specific user. Water dispenser 20 is able to establish a user-specific dietary supplement additive profile in conjunction with a dedicated application to provide the dietary supplement solution, in a similar manner as described hereinabove with respect to water dispenser 10.

Dietary supplement additive unit 215 can be controlled to dispense a customized composition and dose of a dietary supplement solution by a variety of means such as an electronic means, an electro-mechanic means, a pneumatic mechanism, or any other suitable means. In this embodiment, the dispensing of a customized composition and dose of a dietary supplement solution involves pneumatic means.

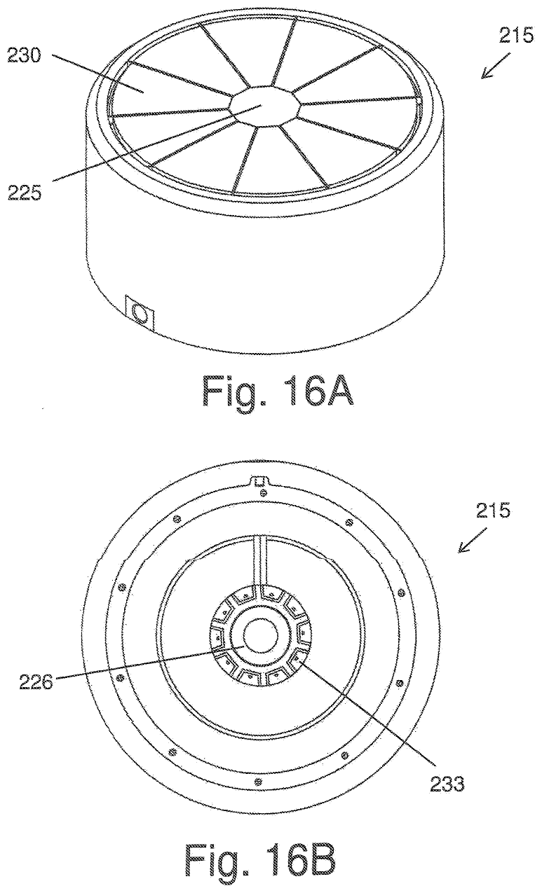

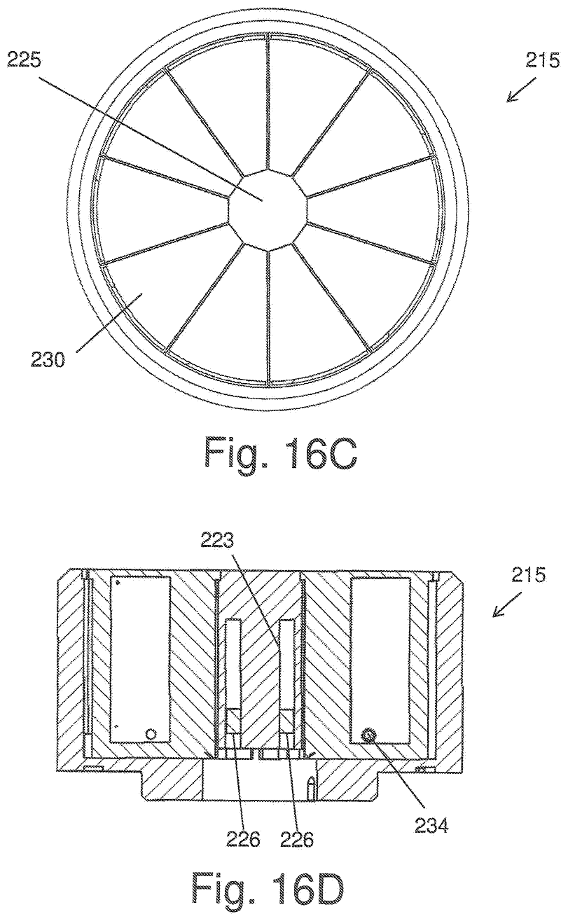

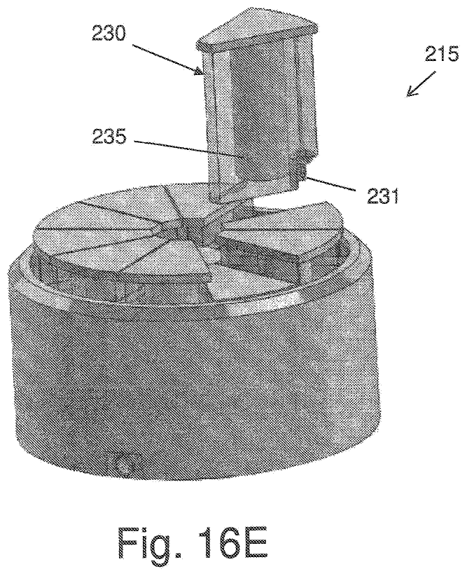

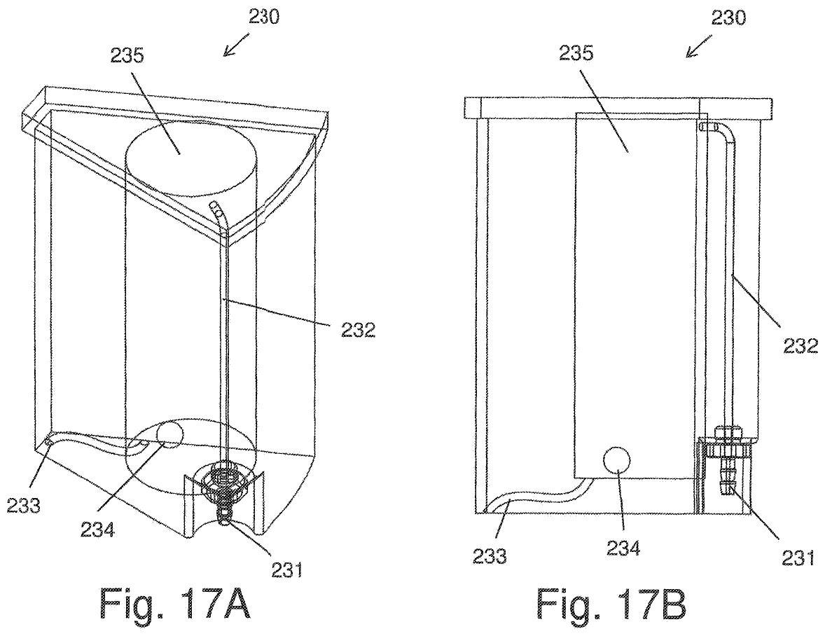

Water dispenser 20 may comprise a compressor 213, a water tank 221, a water filter 203, an Ultra Violet (UV) light 222, a folding tray 212 for a cup 209 as well as any other elements that can be found in countertop water dispensers available in the market, such as The Strauss WaterBars that combines filtration and UV purification filtered process. In addition to such elements, water dispenser 20 comprises an electric faucet 224 that is adapted to control a pneumatic entry 231 (see FIGS. 16E, 17A and 17B) of a vitamin capsule 230, a display unit 210, an RFID reader 225 (e.g., that may read a barcode of vitamin capsule 230).

FIGS. 16A-16E illustrate dietary supplement additive unit 215 of water dispenser 20, according to an embodiment of the invention. Dietary supplement additive unit 215 comprises ten units of vitamin capsule 230; each capsule has the same structure but each may contain a different content. FIGS. 17A-17B illustrate in further details the structure of vitamin capsule 230. Each capsule 230 comprises a vitamin container 235, the pneumatic entry 231 that is in fluid communication with tube 232 that ends at an inlet at an upper section of vitamin capsule 230, a secondary magnet 234, and a liquid outlet 233.

Secondary magnet 234 loosely sits at the bottom of vitamin container 235 and a permanent main magnet 226 which is fixedly housed within a magnet house 223 of the dietary supplement additive unit 215 and adapted to be magnetically coupled with the secondary magnet 234 instantaneously rotated to a given region of the vitamin container 235, causing the secondary magnet 234 to be rotatably displaced within the vitamin container 235 by an agitating action.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1. A dispenser for dispensing an individualized amount of dietary supplements, comprising:
a) a plurality of replaceable cartridges, each of which containing a dietary supplement solution and at least two of which containing different dietary supplement solutions;
b) a discharge port of the dispenser;
c) a control system for controllably discharging a predefined dose of a dietary supplement solution, upon demand, from at least one of said cartridges to said discharge port; and
d) means for preventing settling of dietary supplement particles within each of said cartridges.

2. The dispenser according to claim 1, further comprising a corresponding injector member associated with each of the cartridges for causing discharge of the predefined dose of dietary supplement solution to the discharge port and which is in data communication with the control system.

3. The dispenser according to claim 2, wherein each of the cartridges is fitted with a substantially vertically oriented cylindrical barrel prefilled with a specific dietary supplement solution, and the corresponding injector member comprises a piston that is sealingly engaged with the barrel, a plunger connected to the piston, and an electromechanical actuator positioned above the barrel for causing plunger displacement.

4. The dispenser according to claim 3, wherein the electromechanical actuator is a stepper motor in data communication with the control system and the plunger is a plunger rod downwardly extending from a drive shaft of the stepper motor with which it is connected to the piston, the corresponding injector member further comprising a cylinder surrounding, and in threaded engagement with, the plunger rod to thereby facilitate limited and determinable rotational displacement of the plunger rod and corresponding discharge of the predefined dose of the dietary supplement solution in response to plunger rod rotation.

5. The dispenser according to claim 2, wherein the means for preventing settling of dietary supplement particles within each of said cartridges comprises stirring means.

6. The dispenser according to claim 5, wherein the stirring means comprises a rotatable dietary supplement additive unit provided with the plurality of cartridges, such that the centrifugal force acting on all of said cartridges during rotation of said dietary supplement additive unit causes the dietary supplement solution to be sufficiently stirred to prevent settling of the dietary supplement particles.

7. The dispenser according to claim 6, wherein the dietary supplement additive unit comprises—
i. a circular base that is rotatable about a central vertical axis;
ii. an electrical motor in driving engagement with said base;
iii. the plurality of cartridges which are secured to said base and are circumferentially spaced from each other; and
iv. the corresponding injector member associated with each of the cartridges.

8. The dispenser according to claim 3, wherein a bottom end of the barrel has lower tapered surfaces that taper into a hollow tip from which a conduit extends radially inwardly to the discharge port, such that the predefined dose of the dietary supplement solution is urged to flow through the barrel, conduit and discharge port.

9. The dispenser according to claim 8, wherein the means for preventing settling of dietary supplement particles within each of said cartridges further comprises a metallic ball that loosely sits on top of the hollow tip and a permanent magnet which is fixedly housed within a casing of the dispenser and adapted to be magnetically coupled with the metallic ball of the cartridge instantaneously rotated to a given region of the casing, causing the metallic ball to become unseated from the hollow barrel tip and also to be rotatably displaced within the barrel by an agitating action.

10. The dispenser according to claim 1, which is a water dispenser, the dispenser further comprising watery delivery apparatus for timely delivering a supply of potable water to the discharge port so that said water supply will be combined with the predefined dose of dietary supplement.

11. The dispenser according to claim 10, further comprising temperature control apparatus for regulating the temperature of the supply of water.

12. The dispenser according to claim 10, further comprising means for submitting a request to the control system for a desired dietary supplement transfer operation by which a predefined dose of each of a combination of dietary supplement solutions is discharged to the discharge port.

13. The dispenser according to claim 12, wherein the means for submitting a request to the control system is a touch screen.

14. The dispenser according to claim 12, wherein the means for submitting a request to the control system comprises communication apparatus, the communication apparatus configured to receive the request by a signal transmitted from a dedicated application running on a processor of a computerized device.

15. The dispenser according to claim 1, wherein that dietary supplement particles that are prevented from settling within each of the cartridges is powder that is maintained as a dispersed suspension within the dietary supplement solution.

16. The dispenser according to claim 2, wherein each of the cartridges for causing discharge of the predefined dose of dietary supplement solution to the discharge port is driven by a pneumatic means.

17. The dispenser according to claim 16, wherein the pneumatic means are controlled by an electric faucet.
